(19) United States
(12) Patent Application Publication
(10) Pub. No.: US 2015/0125874 A1
(43) Pub. Date: May 7, 2015

(54) TRANSIENT FLOW ASSAY

(71) Applicant: PRESIDENT AND FELLOWS OF HARVARD COLLEGE, Cambridge, MA (US)

(72) Inventors: Anand Bala Subramaniam, Cambridge, MA (US); George M. Whitesides, Newton, MA (US); Olga Taran, Brookline, MA (US); Elizabeth Jane Maxwell, Ottawa (CA)

(21) Appl. No.: 14/398,327

(22) PCT Filed: May 2, 2013
(86) PCT No.: PCT/US2013/039167

§ 371 Date: Oct. 31, 2014

Related U.S. Application Data
(60) Provisional application No. 61/641,442, filed on May 2, 2012.

(51) Int. Cl. G01N 33/53; G01N 15/02; G01N 15/06

(52) U.S. Cl. CPC G01N 33/53; G01N 15/06; G01N 15/02; G01N 2015/1087
USPC 435/7.1; 436/501; 435/29; 73/865.5

(57) ABSTRACT

Described herein are assays for determining total suspended solids (TSS) in liquids. Here TSS can be determined by flowing turbid liquid samples in a porous medium. Using such an assay, TSS can be determined with small volumes of liquid and in short times without the need for dedicated optics and instruments. The assays can be used to determine total suspended solids in any liquid medium, for example, the assay can be used in an immunoprecipitin assay to determine the amount of antigen or antibody present in blood or other fluid.

### CROSS-REFERENCE TO RELATED APPLICATION

This application claims benefit under 35 U.S.C. §119(e) of U.S. Provisional Application No. 61/641,442 filed May 2, 2012, the contents of which are incorporated herein by reference in its entirety.

### TECHNICAL FIELD

The present disclosure relates generally to devices and methods for determining the amount and/or concentration of solids suspended in a liquid medium.

### BACKGROUND

Determining the concentration of suspended solids is important for industry, e.g., beverage (clarity of wine, beer, juice, is a function of suspended solids) effluents (EPA mandates the amount of suspended solids allowed in water entering a river), water quality (determining the amount of suspended solids in rivers, lakes and oceans is a metric of pollution), medical diagnostic tests (measuring the amount of precipitation in a mixture of antigens and antibodies is a means of determining general health, diagnosing disease and determining if immunizations have been effective), and biotechnology (bacteria and virus growth curves in large scale bioreactors.

Determining the total suspended solids (TSS) in liquids is key requirement in many chemical engineering applications. For example, TSS in effluents is a parameter set by the Environmental Protection Agency (EPA) and the clarity of beverages is determined by the amount of residual solids suspended in the broth. Furthermore, measuring the amount of solid precipitates formed when antigens react with antibodies is a foundational principle used to conduct a wide variety of sensitive immunoassays. Typically, TSS is determined by measuring the light scattered off the suspended solids. To obtain accurate measurements with existing technology, well-defined light sources and detectors are required, and thus dedicated and well-calibrated instruments are essential. For example, extant methods to measure solids suspended in liquids employ light scattering. For immunoassays, diffusion of antibodies and antigens in a gel is used as a quantification mechanism.

Accordingly, there is need in the art for methods for TSS determination without requiring dedicated or expensive instruments.

### SUMMARY

In one aspect provided herein is a low-cost, simple assay for quantifying the amount of solids suspended in a liquid medium. In some embodiments, the assay comprises flowing a turbid liquid sample in a porous medium and measuring the transient flow front generated by the turbid liquid sample in the porous medium over a period of time. The assay can be performed without the need for dedicated optics and instruments

In some embodiments, total suspended solids content of a liquid can be determined with small volumes, e.g., about 20 μl, and short time periods, e.g., about 1 minute or less.

In some embodiments, the assay can be used in an immunoprecipitin assay to determine the amount of antigens or antibodies, (e.g., serum IgG) present in a liquid sample such as blood or other biological sample.

In another aspect, provided herein is kit for determining TSS in a liquid sample. In some embodiments, the kit comprises a solid substrate. The substrate is coated with a layer of porous material and the porous material coating layer is coated with a layer of liquid impermeable material having one or more pores.

### BRIEF DESCRIPTION OF THE DRAWINGS

FIG. 1 is a schematic representation of an embodiment of the assay described herein. A drop of solution containing the solids is applied to a TLC plate sealed with packing tape, via a small hole in the tape. The solution is allowed to permeate the porous substrate for a short period of time (e.g., 20 seconds). The fluid front that is generated due to the transient flow, e.g., radial diffusion, is measured.

FIG. 2A is a schematic representation showing maximum precipitation occurs at equivalence point of antigen and antibody concentration.

FIG. 2B is a line graph showing addition of serially diluted antigen samples to solutions with fixed antibody concentration results in a bell-shaped curve when samples are assayed for turbidity.

FIGS. 3A and 3B show that amount of antigen present in a sample can be quantified by an assay described herein, also referred to as a transient flow immune assay (TrIA). FIG. 3A shows transient flow from liquid samples that were assayed through turbidimetry. Four 20 μl aliquots of the samples were placed on tape-covered alumina TLC plates and allowed to infiltrate the porous matrix for 20 seconds. Then excess liquid was wiped off the tape. The liquid phase was labeled with fluorescein which was used as a marker for the fluid front. The fluorescence signal of fluorescein was then read on a fluorescence scanner. FIG. 3B is a line graph showing that the diameter of the spreading front correlated with the concentration of antigen added and the bell-shaped curve characteristic of immunoprecipitation reactions is clearly observed. TrIA gives an equivalence antigen concentration of 0.0675 mg/ml which matched the value obtained through turbidimetry (FIG. 2B).

FIG. 4 is schematic representation of an approach to the assay.

FIGS. 5A and 5B show that low volume fraction suspensions can be determined using an embodiment of the assay described herein. FIG. 5A shows diameters of transient flow. Concentrations are 1 w/v %, 0.1 w/v %, 0.01 w/v %, and 0.001 w/v % from top to bottom. FIG. 5B shows that diameter of transient flow correlates with the amount of solid in the liquid. As can be seen, the lower the amount of solid in the sample larger the diameter of transient flow.

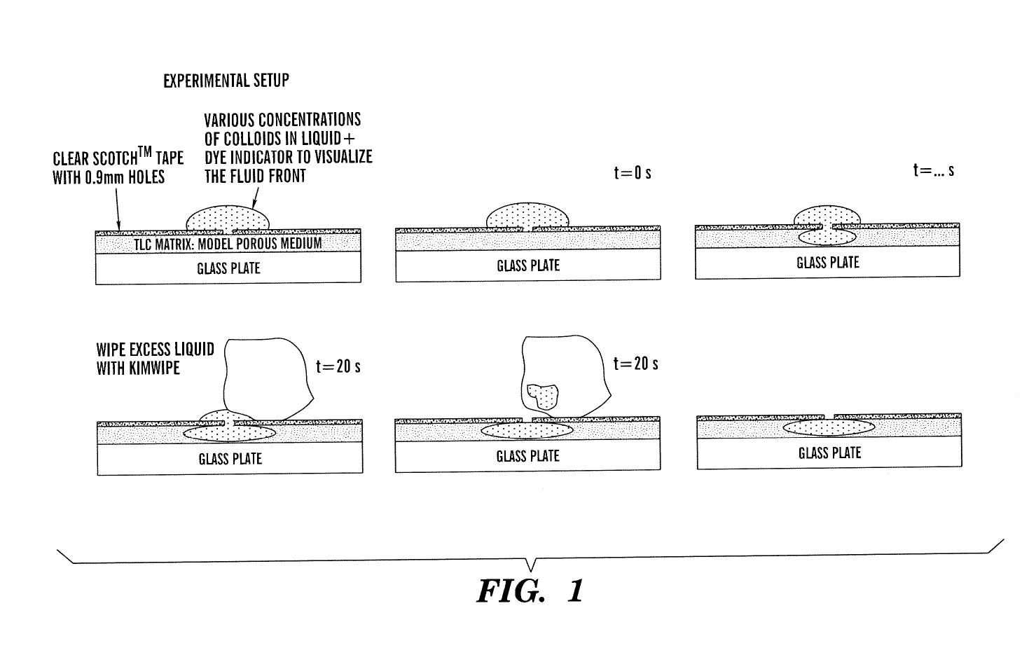

### DETAILED DESCRIPTION OF EXEMPLARY EMBODIMENTS

Aspects of the assay described herein are based on the inventors' discovery of a new physical phenomenon that the clogging of solids in porous medium can be used to quantify total suspended solids in liquids. The inventors have discovered that the clogging causes a transient flow in the porous medium which has particular properties which correlate with the amount of solids present in the liquid. The amount of solids in a liquid can be measured visually and cheaply. The inventors have discovered that the diameter of fluid front in the porous medium and concentration of solids in the liquid sample can be correlated and the diameter is proportional to the amount of suspended solid present in the sample.

The assay can be used to determine the concentration of colloidal solids, i.e. small particles (for example inorganic chemical precipitates, soil, clays, organic particles such as bacteria, viruses, precipitates of biomolecules such as DNA, RNA and proteins etc. . . . ) in a liquid. Generally, the assay comprises flowing a liquid containing the suspended particles through a porous medium and measuring the size of the liquid front, e.g., radial diffusion of the liquid in the porous medium. Without limitations, the assay described herein can be performed without specialized optics and lasers to perform light scattering measurements to determine the concentration of solids in any liquid sample. Further, the assay can be performed by simple visual measurements on samples and provides a permanent visual indicator of the amount of suspended solids in a liquid.

Additionally, since the diameter of the liquid front is proportional to the antigen concentration in a sample, e.g., blood, the assay can be used to quantify the amount of antigens present in a liquid sample thus conducting an immunoassay.

Unless defined otherwise, all technical and scientific terms used herein have the same meaning as those commonly understood to one of ordinary skill in the art to which this invention pertains. Although any known methods, devices, and materials can be used in the practice or testing of the invention, the methods, devices, and materials in this regard are described herein.

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein the terms “comprising” or “comprises” means “including” or “includes” and are used in reference to compositions, methods, and respective component(s) thereof, that are useful to the invention, yet open to the inclusion of unspecified elements, whether useful or not.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean±5% of the value being referred to. For example, about 100 means from 95 to 105.

Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

In some embodiments, the method comprises applying the liquid containing the suspended particles to a porous medium and measuring size of liquid front in the porous medium after a predetermined time. The size of the liquid front can be the diameter of radial diffusion of the liquid in the porous medium. In some embodiments, one or more dilutions of the test sample can be applied to the porous medium.

The liquid comprising the suspended particle can be any flowable material that comprises the suspended particle. Without wishing to be bound by theory, the liquid can be aqueous or non-aqueous, supercritical fluid, gases, solutions, suspensions, and the like.

In some embodiments, the liquid comprising the suspended particles is a biological fluid. The terms “biological fluid” and “biofluid” are used interchangeably herein and refer to aqueous fluids of biological origin, including solutions, suspensions, dispersions, and gels, and which may or may not contain undissolved particulate matter. Exemplary biological fluids include, but are not limited to, blood (including whole blood, plasma, cord blood and serum), lactation products (e.g., milk), amniotic fluids, peritoneal fluid, sputum, saliva, urine, semen, cerebrospinal fluid, bronchial aspirate, perspiration, mucus, liquefied feces, synovial fluid, lymphatic fluid, tears, tracheal aspirate, and fractions thereof. Another example of a group of biological fluids are cell culture fluids, including those obtained by culturing or fermentation, for example, of single- or multi-cell organisms, including prokaryotes (e.g., bacteria) and eukaryotes (e.g., animal cells, plant cells, yeasts, fungi), and including fractions thereof. Yet another example of a group of biological fluids are cell lysate fluids including fractions thereof. Still another example of a group of biological fluids are culture media fluids including fractions thereof. For example, culture media comprising biological products (e.g., proteins secreted by cells cultured therein) can be collected and amount of suspended solids can be determined by the assay described herein.

In some embodiments, the liquid comprising the suspended particles is a non-biological fluid. As used herein, the term “non-biological fluid” refers to any aqueous, non-aqueous or gaseous sample that is not a biological fluid as the term is defined herein. Exemplary non-biological fluids include, but are not limited to, water, salt water, brine, organic solvents such as alcohols (e.g., methanol, ethanol, isopropyl alcohol, butanol etc. . . . ), saline solutions, sugar solutions, carbohydrate solutions, lipid solutions, nucleic acid solutions, hydrocarbons (e.g. liquid hydrocarbons), acids, gasolines, petroleum, liquefied samples (e.g., liquefied foods), gases (e.g., oxygen, CO₂, air, nitrogen, or an inert gas), and mixtures thereof.

In some embodiments, the source fluid is a media or reagent solution used in a laboratory or clinical setting, such as for biomedical and molecular biology applications. As used herein, the term “media” refers to a medium for maintaining a tissue or cell population, or culturing a cell population (e.g. “culture media”) containing nutrients that maintain cell viability and support proliferation. The cell culture medium can contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art. The media can include media to which cells have been already been added, i.e., media obtained from ongoing cell culture experiments, or in other embodiments, be media prior to the addition of cells.

The suspended solids in the liquid can be any particulate matter or any molecule that can turn into a suspended solid under assay conditions. For example, a multivalent affinity molecule can be added to the liquid comprising the molecule. Binding of the affinity molecules to the molecule of interest can lead to formation of a network or lattice of molecules of interest and affinity molecules which can be unsoluble in the liquid. Since the diameter of the liquid front is proportional to the amount of the suspended solids in the sample, the assay can be used to quantify the amount of a molecule in the sample by converting the molecule into a suspended solid. Accordingly, the assay described herein can also be used to determine the amount or concentration of a molecule or target of interest in the sample.

As used herein, the term “affinity molecule” refers to any molecule that is capable of specifically interacting or binding with another molecule, i.e. a target molecule. Generally, the nature of interaction or binding between the affinity molecule and the target molecule is non-covalent, such as one or more of hydrogen bonding, Van der Waals forces, electrostatic forces, hydrophobic forces, and the like. However, interaction or binding can also be covalent.

An affinity molecule can be a naturally-occurring, recombinant or synthetic molecule. However, an affinity molecule need not comprise an entire naturally occurring molecule but can consist of only a portion, fragment or subunit of a naturally or non-naturally occurring molecule. Exemplary affinity molecules include, but are not limited to, ligand receptors, ligands for a receptor, one member of a coupling pair, nucleic acids (e.g., aptamers), peptides, proteins, peptidomimetics, antibodies, portion of an antibody, antibody-like molecules, antigens, and the like.

In some embodiments, the affinity molecule is an antibody or a portion thereof. In some embodiments, the affinity molecule is an antigen binding fragment of an antibody. As used herein, the term “antibody” or “antibodies” refers to an intact immunoglobulin or to a monoclonal or polyclonal antigen-binding portion with the Fc (crystallizable fragment) region or FcRn binding fragment of the Fc region. The term “antibodies” also includes “antibody-like molecules”, such as portions of the antibodies, e.g., antigen-binding portions. Antigen-binding poritons can be produced by recombinant DNA techniques or by enzymatic or chemical cleavage of intact antibodies. “Antigen-binding portions” include, inter alia, Fab, Fab′, F(ab′)2, Fv, dAb, and complementarity determining region (CDR) fragments, single-chain antibodies (scFv), single domain antibodies, chimeric antibodies, diabodies, and polypeptides that contain at least a portion of an immunoglobulin that is sufficient to confer specific antigen binding to the polypeptide. Linear antibodies are also included for the purposes described herein. The terms Fab, Fc, pFc′, F(ab′) 2 and Fv are employed with standard immunological meanings (Klein, Immunology (John Wiley, New York, N.Y., 1982); Clark, W. R. (1986) The Experimental Foundations of Modern Immunology (Wiley & Sons, Inc., New York); and Roitt, I. (1991) Essential Immunology, 7th Ed., (Blackwell Scientific Publications, Oxford)). Antibodies or antigen-binding portions specific for various antigens are available commercially from vendors such as R&D Systems, BD Biosciences, e-Biosciences and Miltenyi, or can be raised against these cell-surface markers by methods known to those skilled in the art.

An affinity molecule can be generated by any method known in the art. For example, antibodies can be found in an antiserum, prepared from a hybridoma tissue culture supernatant or ascites fluid, or can be derived from a recombinant expression system, as is well known in the art. Fragments, portions or subunits of e.g., an antibody, receptor or other species, can be generated by chemical, enzymatic or other means, yielding for example, well-known (e.g., Fab, Fab′) or novel molecules. The present invention also contemplates that affinity molecules can include recombinant, chimeric and hybrid molecules, such as humanized and primatized antibodies, and other non-naturally occurring antibody forms. Those skilled in the art will recognize that the non-limiting examples given above describing various forms of antibodies can also be extended to other affinity molecules such that recombinant, chimeric, hybrid, truncated etc., forms of non-antibody molecules can be used in the compositions and methods of the present invention.

In some embodiments, the affinity molecule can be conjugated with a detectable label. Detectable labels are described in more detail below.

In one embodiment, the assay can be used to determine the amount of an antigen in a liquid sample, e.g., blood. Antibody or antibodies which can bind with the antigen can be added to the sample. The sample can then be applied to the porous medium and the size or distance of the liquid front measured. The size of the liquid front can be compared to a reference or standard sample and amount of the antigen determined in the original sample.

The liquid comprising the suspended particles can be allowed to flow in the porous medium for any desirable time before measuring the size of the liquid front. Generally, the liquid can be allowed to flow for about 5 hours or less before the liquid front is measured. In some embodiments, liquid can be allowed to flow for a period of about 2 hours, about 1.5 hours, about 1 hour, about 45 minutes, about 30 minutes, about 25 minutes, about 20 minutes, about 15 minutes, about 14 minutes, about 13 minutes, about 12 minutes, about 11 minutes, about 10 minutes, about 9 minutes, about 8 minutes, about 7 minutes, about 6 minutes, about 5 minutes, about 4 minutes, about 3 minutes, about 2 minutes, about 1 minute, about 45 seconds, about 30 seconds, about 25 seconds, about 20 seconds, about 15 seconds, or about 10 seconds or less before the liquid front is measured.

After the liquid comprising the suspended particles has been allowed to flow in the porous medium for the desired period of time, any excess liquid, which did not diffuse into the porous medium, can be removed before measuring the size of the liquid front. This can be accomplished, for example, by wiping the surface of the porous medium or blowing air over the porous material.

As used herein, the terms “porous medium” or “porous media” include, but are not limited to, an article that has a porous structure and is capable of absorbing a liquid. As such, the term “porous medium” includes any material in which a fluid can diffuse from the exterior into the interior thereof, or vice versa. It includes, but is not limited to media used for filtration, adsorption, absorption, or other forms of separation and/or removal of selected components from a liquid. Without limitations, any material having a porous nature can be used in the assay described herein. Examples of porous media include, but are not limited to, thin layer chromatography (TLC) plates; paper; clay backed sheets; packed beds; membranes, such as fibrous screen membranes, cast membranes and track-etched membranes; sintered porous polymeric media; and media with polymeric matrices with or without a particulate contained therein. Porous media include media used to separate blood components or blood fractions such as plasma from whole blood, or remove leukocytes from blood, or remove bacteria or other pathogens from a biological fluid, or remove compounds used in or resulting from a pathogen inactivation treatment from biological fluid. As defined, “porous medium” or “porous media” is not limited to membranes, polymeric filtration or removal media, but can include materials as diverse as wood, paper, concrete and the like. Without limitations, the porous medium can be in any shape or form. For example, porous medium can be in the form of a film, plate, well, bead, membrane, and the like.

The porous material can be coated with a liquid impermeable layer. One or more pores or holes in the liquid impermeable layer can be used to apply the liquid to the porous material. Without limitation, the pore or the hole in the liquid impermeable layer allows one to apply the liquid to a predefined size of the porous material. The liquid impermeable layer can comprise any material that can provide a nonporous medium when coated on the porous medium. In one example non-limiting example, the liquid impermeable layer can be a layer of SCOTCH™ tape.

For quantitation of the solids suspended in a liquid, size of the liquid front on the porous material can be correlated with a standard or reference sample. For example, samples comprising known amount of solid content can be applied to the porous material along with the test sample. After measuring the size of the liquid fronts for the reference or standard samples, a standard curve can be generated correlating the size of the liquid front to the amount of solid in the reference or standard samples. Amount of the solid in the test sample then can be determined from the standard curve.

In some embodiments, the porous medium comprises a solid substrate coated with a layer of a porous material. As used herein, the term “porous material” refers to any material capable of providing lateral flow. This can include material such as nitrocellulose, nitrocellulose blends with polyester or cellulose, untreated paper, porous paper, rayon, glass fiber, acrylonitrile copolymer or nylon. One skilled in the art will be aware of other porous materials that allow lateral flow. The term “lateral flow” refers to liquid flow in which all of the dissolved of dispersed components of the liquid are carried at substantially equal rates and with relatively unimpaired flow laterally through the material, as opposed to preferential retention of one or more components as would occur, e.g., in materials capable of adsorbing or imbibing one or more components.

Any method known in the art for detecting or measuring a liquid front can be used. Exemplary methods include, but are not limited to, visual, spectrometry, fluorometry, imaging, microscopy imaging, immunoassay, and the like. Additionally, detecting or measuring of liquid front can be performed via automated image acquisition and analysis.

In some embodiments, prior to measuring the liquid front, the liquid front can be stained with a detectable label. The detectable label can be added to the liquid before application onto the porous medium or after the liquid has been applied to the porous medium. As used herein, the term “detectable label” refers to a composition capable of producing a detectable signal. Detectable labels include any composition detectable by spectroscopic, photochemical, biochemical, immunochemical, electrical, optical or chemical means. Suitable labels include dyes, visual dyes, fluorescent molecules, radioisotopes, nucleotide chromophores, enzymes, substrates, chemiluminescent moieties, bioluminescent moieties, and the like. As such, a label is any composition detectable by visual, optical, spectroscopic, photochemical, biochemical, immunochemical, electrical, or chemical means needed for the methods and devices described herein.

Examples of visual dyes include soluble visual dyes such as solvent dyes, pigments, sulfur dyes, mordant dyes, and species such as fluorescein, rhodamine and derivatives (such as sulfurrhodamine, rhodamine-hydride, and rhodamine hydrazide), oxazine dyes, cyanine dyes, and azol dyes. Specific examples of suitable dyes include, but are not limited to, Texas Red hydrazine, Congo Red, Trypan Blue, Lissamine Blue, Remazol Black, Remazol Brilliant Red, Rhodamine β Isotlliocyanate, Cy5-Osu mono functional reactive dye, Reactive Orange 16, Uniblue A, and the like.

A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound. Exemplary fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca2+ Dye; Calcium Green-2 Ca2+; Calcium Green-5N Ca2+; Calcium Green-C18 Ca2+; Calcium Orange; Calcofluor White; Carboxy-X-rhodamine (5-ROX); Cascade Blue™; Cascade Yellow; Catecholamine; CFDA; CFP—Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hcp; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; SgBFP™; SgBFP™ (super glow BFP); SgGFP™; SgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamineIsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; and YOYO-3. Many suitable forms of these fluorescent compounds are available and can be used.

In some embodiments, the detectable label is a fluorophore or a quantum dot. Without wishing to be bound by a theory, using a fluorescent reagent can reduce signal-to-noise thus maintaining sensitivity.

Other exemplary detectable labels include radiolabels (e.g., ³H, ¹²⁵I, ³⁵S, ¹⁴C, or ³²P), and colorimetric labels such as colloidal gold or colored glass or plastic (e.g., polystyrene, polypropylene, and latex) beads. Patents teaching the use of such labels include U.S. Pat. Nos. 3,817,837, 3,850,752, 3,939,350, 3,996,345, 4,277,437, 4,275,149, and 4,366,241, content of each of which is incorporated herein by reference in its entirety.

Means of detecting such labels are well known to those of skill in the art. Thus, for example, radiolabels can be detected using photographic film or scintillation counters, fluorescent markers can be detected using a photo-detector to detect emitted light.

Any method known in the art for detecting the particular label can be used for detection. Exemplary methods include, but are not limited to, visual, spectrometry, fluorometry, microscopy imaging, immunoassay, and the like.

### Kits

In another aspect, provided herein are kits for determining the amount of suspended solid in liquid sample. Such kits can be prepared from readily available materials and reagents. For example, such kits can comprise any one or more of the following materials or components, a porous medium, wherein the porous medium comprises a solid substrate coated with a layer of a porous material and the porous material layer is coated with a liquid impermeable layer, wherein the liquid impermeable layer comprises a pore; a dye; an affinity molecule; a reference or standard sample comprising a liquid with known amount of suspended solid.

A variety of kits and components can be prepared for use in the assays described herein, depending upon the intended use of the kit, the particular liquid sample, the particular molecule of interest in the liquid sample, and the needs of the user.

The disclosure is further illustrated by the following examples which should not be construed as limiting. The examples are illustrative only, and are not intended to limit, in any manner, any of the aspects described herein. The following examples do not in any way limit the invention.

### EXAMPLES

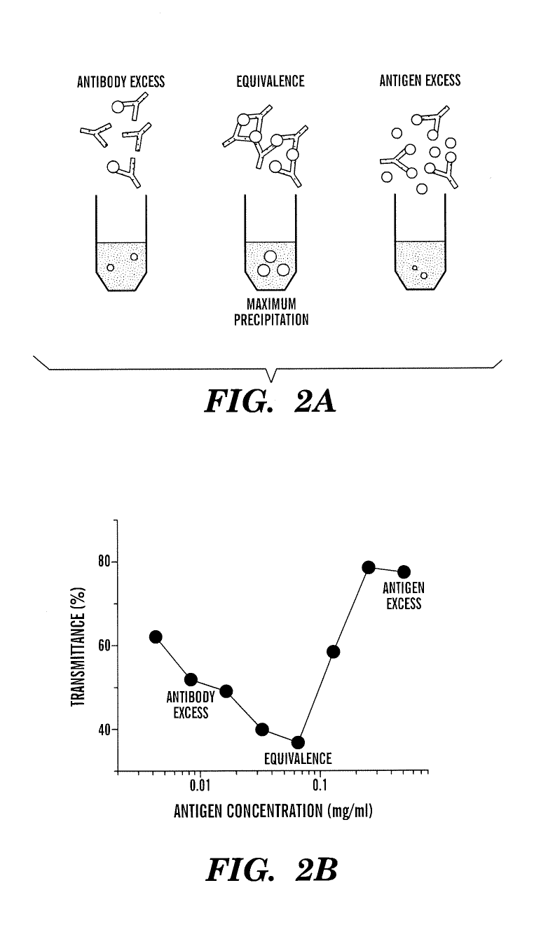

When generic antigens and antibodies are mixed, antibodies bind specifically to their complementary antigens. Since antibodies are polyvalent, at appropriate concentrations of antigens (termed the equivalence region), large lattices of antigen and antibodies form and precipitate out of solution¹⁻³. If the antigen concentration is lower or higher than the equivalence concentration, the amount of precipitates formed is smaller (FIG. 2A). Thus, measuring the amount of precipitate can be a means to determine the concentration of antigens present in solution. Immunoassays based on this general property of antigens and antibodies are called immunoprecipitin or coagulation assays. Immunoprecipitin reactions can be assayed in gels, which are slow, requiring 24 to 48 hours to get a result¹⁻¹², or through light scattering which is rapid, but suffers from the drawback of being expensive and requiring dedicated equipment¹³⁻¹⁹. In this experiment, the inventors demonstrate that a particular aspect of the fluid flow of immunoprecipitate laden liquid samples on aluminum oxide thin layer chromatography (TLC) plates can be used to conduct immunoassays.

IgG from rabbit serum (reagent grade >95%, salt free lyophilized powder) and Anti-Rabbit IgG (whole molecule), antibody produced in goat (whole antiserum), and PEG 6000 (Polyethylene Glycol were purchased from Sigma-Aldrich. Antibodies were stored at 8° C. A 10×PBS solution (Phosphate buffer saline pH 7.0) was purchased from Lonza. All reagents were used without any further treatment.

The inventor prepared a solution of PEG in PBS buffer. PEG concentration (in weight percent) was two times larger than used in the experiments. To avoid interference by suspended solid impurities, the inventors filtered the solution with 0.45 μm filter and stored the solution at room temperature during all the time of the experiment.

A 2 mg/mL Rabbit IgG solution in 0.1M PBS buffer pH 7 was prepared. Anti rabbit IgG stock solution (17.0 mg/mL) was diluted with PEG buffer to 1 mg/mL concentration. Protein solutions were used immediately after preparation.

A conventional precipitin analysis was performed by mixing in 96-well plate 100 μL of Ab with 100 μL IgG solution, which was serially diluted 1:1 with 0.1M PBS buffer pH 7. The reaction mixture was left overnight at 8° C. Precipitin formation was confirmed by turbidity measurements at 550 nm.

As shown in FIG. 2A, for low antigen concentrations there are excess antibodies in solution and small or no precipitates are formed. As antigen concentration is increased, a zone of maximum precipitation is reached where the antigen to antibody concentration is about equal and large precipitate lattices are formed. As antigen concentration is increased further, all the binding sites of the antibodies are taken up and the amount of precipitation again falls. The amount of precipitate formed can be determined through turbidity measurements in a spectrophomoteter. At the equivalence point the amount of light transmitted is minimum, i.e. the sample is most turbid. Turbidimetry gives an equivalence antigen concentration of 0.0675 mg/ml (FIG. 2B). This is the standard assay in the literature.

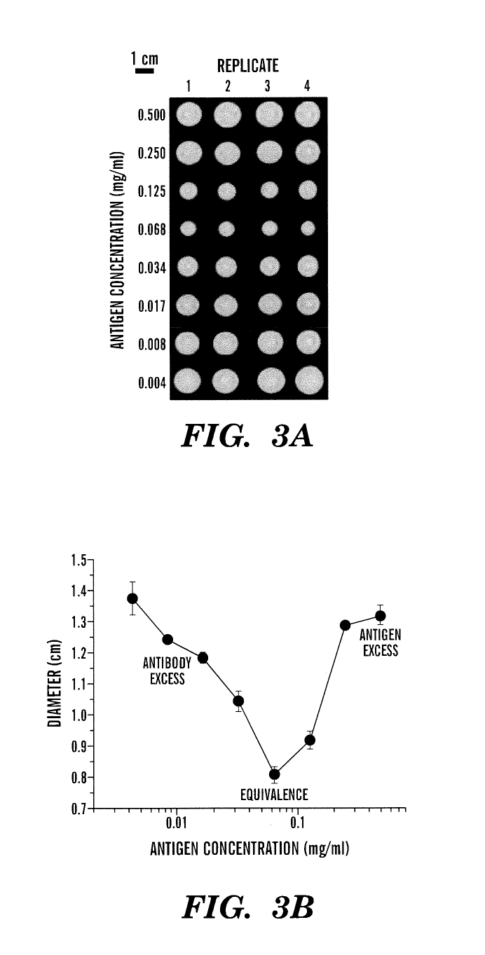

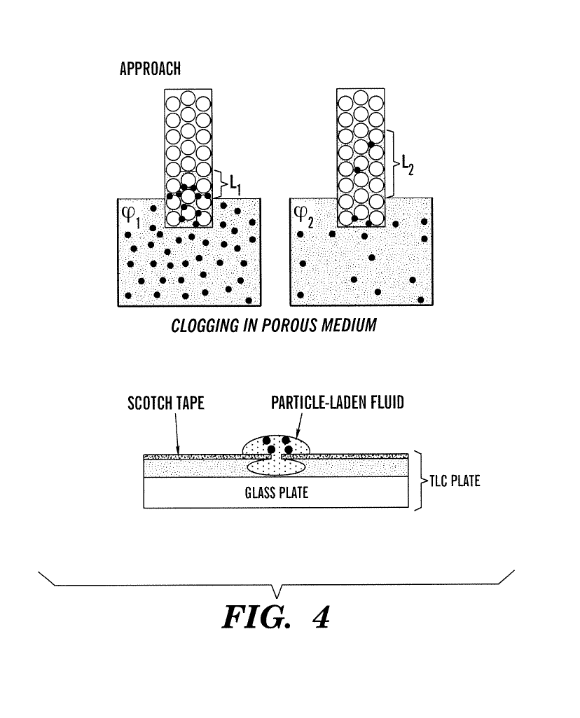

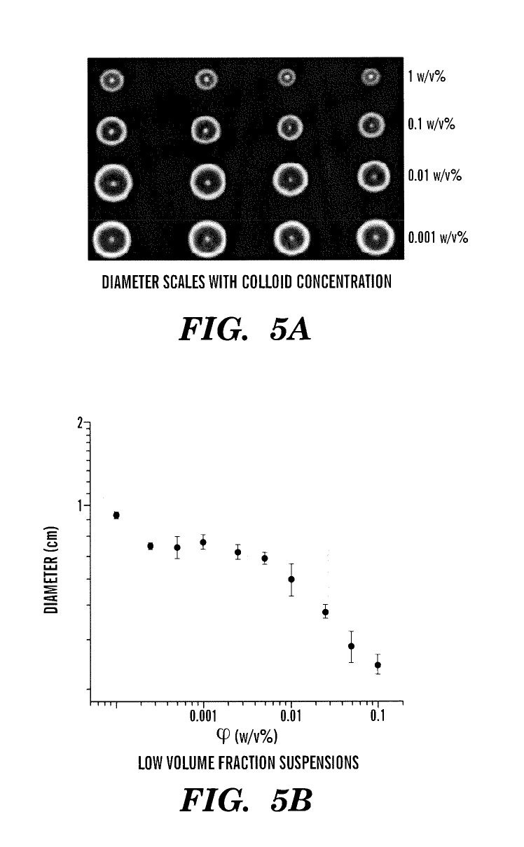

As shown in FIGS. 3A and 3B, amount of antigen present in a sample can be quantitated by an embodiment of the assay described herein. As can be seen from FIGS. 3A and 3B the diameter of the liquid front was proportional to the antigen concentration in the sample. This enabled quantification of the amount of antigens present in the liquid sample Four 20 μl aliquots of the samples that were assayed through turbidimetry were placed on tape-covered alumina TLC plates and allowed to infiltrate the porous matrix for 20 seconds. Excess liquid was wiped off the tape. The liquid phase was labeled with fluorescein which was used as a marker for the fluid front. The fluorescence signal of fluorescein was then read on a fluorescence scanner. The diameter of radial diffusion correlated with the amount of antigen present in the sample (FIG. 3B). The diameter of the spreading front correlated with the concentration of antigen added (FIG. 3A) and the bell-shaped curve characteristic of immunoprecipitation reactions was observed (FIG. 3B).

Since the diameter of the liquid front was proportional to the antigen concentration in the sample the inventors quantified the amount of antigens present in the liquid sample, thus conducting an immunoassay. The assay gave an equivalence antigen concentration of 0.0675 mg/ml which matched the value obtained through turbidimetry (FIG. 2B). This demonstrated that the assay described herein is fast and cheap and can be used without the need for dedicated optics or lasers.

Accordingly, provided herein is an assay for determining total suspended solid content of a liquid. The assay can be used to determine the amount and/or concentration of suspended solids in industrial settingbeverage industry: clarity of wine, beer, juice, is a function of suspended solids), effluents (EPA mandates the amount of suspended solids allowed in water entering a river), water quality, determining the amount of suspended solids in rivers, lakes and oceans is a metric of pollution, medical diagnostic tests, measuring the amount of precipitation in a mixture of antigens and antibodies is a means of determining general health, diagnosing disease and determining if immunizations have been effective, biotechnology: bacteria and virus growth curves in large scale bioreactors. The assay makes quantitative diagnostic tests based on immunoprecipitin reactions accessible for resource-poor settings, point of care situations, and offers time savings while retaining simplicity.

### REFERENCES

1 Fahey, J. L. & McKelvey, E. M. QUANTITATIVE DETERMINATION OF SERUM IMMUNOGLOBULINS IN ANTIBODY-AGAR PLATES. J. Immunol. 94, 84-&, (1965).
2 Mancini, G., Carbonara, A. O. & Heremans, J. F. Immunochemical quantitation of antigens by single radial immunodiffusion. Immunochemistry 2, 235-IN236, (1965).
3 Marrack, J. R. & Richards, C. B. LIGHT-SCATTERING STUDIES OF FORMATION OF AGGREGATES IN MIXTURES OF ANTIGEN AND ANTIBODY. Immunology 20, 1019-&, (1971).
4 Crowle, A. J. INTERPRETATION OF IMMUNODIFFUSION TESTS. Annu Rev. Microbiol. 14, 161-176, (1960).
5 Fedorov, A. A., Kurochkin, V. E., Martynov, A. I. & Petrov, R. V. Theoretical and experimental investigation of immunoprecipitation pattern formation in gel medium. J. Theor. Biol. 264, 37-44, (2010).
6 Berne, B. H. DIFFERING METHODOLOGY AND EQUATIONS USED IN QUANTITATING IMMUNOGLOBULINS BY RADIAL IMMUNODIFFUSION—COMPARATIVE EVALUATION OF REPORTED AND COMMERCIAL TECHNIQUES. Clin. Chem. 20, 61-69, (1974).
7 Killings. L m & Savory, J. NEPHELOMETRIC STUDIES OF PRECIPITIN REACTION—MODEL SYSTEM FOR SPECIFIC PROTEIN MEASUREMENTS. Clin. Chem. 19, 403-407, (1973).
8 Creighto. W d, Lambert, P. H. & Miescher, P. A. DETECTION OF ANTIBODIES AND SOLUBLE ANTIGEN-ANTIBODY COMPLEXES BY PRECIPITATION WITH POLYETHYLENE-GLYCOL. J. Immunol. 111, 1219-1227, (1973).
9 Killings. L m & Savory, J. MANUAL NEPHELOMETRIC METHODS FOR IMMUNOCHEMICAL DETERMINATION OF IMMUNOGLOBULINS IGG, IGA, AND IGM IN HUMAN SERUM. Clin. Chem. 18, 335-&, (1972).
10 Harringt. J c, Fenton, J. W. & Pert, J. H. POLYMER-INDUCED PRECIPITATION OF ANTIGEN-ANTIBODY COMPLEXES—PRECIPIPLEX REACTIONS. Immunochemistry 8, 413-&, (1971).
11 Fedorov, A. A., Martynov, A. I., Kurochkin, V. E., Andreev, I. V. & Petrov, R. V. Microstructure of immunoprecipitation patterns in gel: The mechanism of spatial self-organization of interacting protein populations in gel-like media. Dokl. Biochem. Biophys. 421, 207-210, (2008).
12 Virella, G. & Fudenberg, H. H. COMPARISON OF IMMUNOGLOBULIN DETERMINATIONS IN PATHOLOGICAL SERA BY RADIAL IMMUNODIFFUSION AND LASER NEPHELOMETRY. Clin. Chem. 23, 1925-1928, (1977).
13 Lopesvirella, M. F. L., Virella, G., Evans, G., Malenkos, S. B. & Colwell, J. A. IMMUNONEPHELOMETRIC ASSAY OF HUMAN APOLIPOPROTEIN-AL. Clin. Chem. 26, 1205-1208, (1980).
14 Conrad, A., Schurmann, J., Kreutz, F. H. & Sieber, A. ELABORATION OF A METHOD FOR QUANTITATIVE-DETERMINATION OF PROTEINS BY LASER NEPHELOMETRY IN CLINICAL ROUTINE LABORATORY. Journal of Clinical Chemistry and Clinical Biochemistry 16, 299-305, (1978).
15 Sternberg, J. C. RATE NEPHELOMETER FOR MEASURING SPECIFIC PROTEINS BY IMMUNOPRECIPITIN REACTIONS. Clin. Chem. 23, 1456-1464, (1977).
16 Deaton, C. D., Maxwell, K. W., Smith, R. S. & Creveling, R. L. USE OF LASER NEPHELOMETRY IN MEASUREMENT OF SERUM-PROTEINS. Clin. Chem. 22, 1465-1471, (1976).
17 Buffone, G. J., Savory, J. & Hermans, J. EVALUATION OF KINETIC LIGHT-SCATTERING AS AN APPROACH TO MEASUREMENT OF SPECIFIC PROTEINS WITH CENTRIFUGAL ANALYZER 0.2. THEORETICAL CONSIDERATIONS. Clin. Chem. 21, 1735-1746, (1975).
18 Lizana, J. & Hellsing, K. POLYMER ENHANCEMENT OF AUTOMATED IMMUNOLOGICAL NEPHELOMETRIC ANALYSIS, AS ILLUSTRATED BY DETERMINATION OF URINARY ALBUMIN. Clin. Chem. 20, 415-420, (1974).
19 Buffone, G. J., Savory, J. & Cross, R. E. USE OF A LASER-EQUIPPED CENTRIFUGAL ANALYZER FOR KINETIC MEASUREMENT OF SERUM IGG. Clin. Chem. 20, 1320-1323, (1974).

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

What is claimed is:

1. An assay for determining an amount of solid suspended in a liquid sample, the method comprising: applying a liquid sample containing suspended particles to a porous medium; and measuring size or migration of liquid front in the porous medium after a predetermined time relative to a standard or reference sample.

2. The assay of claim 1, comprising further adding an affinity molecule to the liquid sample to form the suspended solid.

3. The assay of claim 2, wherein the affinity molecule is an antibody or antigen-binding portion thereof.

4. A kit comprising a porous material, wherein the porous medium comprises a solid substrate coated with a layer of a porous material and wherein the porous material layer is coated with a liquid impermeable layer and the liquid impermeable layer comprises a pore therein.

5. The kit of claim 4, wherein the kit further comprises a dye, an affinity molecule, a reference or standard sample comprising a liquid with known amount of suspended solid.
